(19) United States
(12) Patent Application Publication
(10) Pub. No.: US 2015/0011446 A1
(43) Pub. Date: Jan. 8, 2015

(54) (PER)FLUOROPOLYETHERS WITH bi- OR ter-PHENYL END GROUPS

(71) Applicant: SOLVAY SPECIALTY POLYMERS ITALY S.P.A., Bollate (IT)

(72) Inventors: Durairaj Baskaran, Knoxville, TN (US); Prakash Wadgaonkar, Pune (IN); Shamal Menon, Katraj (IN); Claudio Adolfo Pietro Tonelli, Paderno d' Adda (IT)

(21) Appl. No.: 14/378,115

(22) PCT Filed: Feb. 12, 2013
(86) PCT No.: PCT/EP2013/052739

§ 371 Date: Aug. 12, 2014

(30) Foreign Application Priority Data
Feb. 17, 2012 (IN) 468/DEL/2012
May 9, 2012 (EP) 12167213.3

(51) Int. Cl. C10M 107/44; C07C 41/30

(52) U.S. Cl. CPC C10M 107/44; C07C 41/30
USPC 508/549; 568/586

(57) ABSTRACT

The present invention relates (per)fluoropolyether compounds comprising a (per)fluoropolyether chain (R_{f}) having at least two chain ends, wherein at least one of said chain ends have a bi- or ter-phenyl group bearing at least one nitro group and, optionally one or more further substituents. The invention also relates to processes for the preparation of such compounds and to their use for the preparation of lubricant compositions in the form of oils or greases.

This application claims priority to Indian patent application IN 468/DEL/2012, filed on 17 Feb. 2012 and European patent application EP 12167213.3, filed on 9 May 2012; the whole content of these applications is incorporated herein by reference for all purposes.

### TECHNICAL FIELD

The present invention relates to fluorinated polymers, in particular to (per)fluoropolyethers to be used as lubricants or as additives for (per)fluorinated oils or greases, and to methods for the manufacture of such (per)fluoropolyethers.

### BACKGROUND ART

Lubricating oils and greases based on (per)fluoropolyethers are usually endowed with high thermal stability; however, when they are heated for prolonged times at temperatures higher than 230° C., partial or total degradation of the polymer chain and formation of volatile compounds occurs; this degradation is usually accompanied by the formation of Lewis acids, due to corrosion of the metallic parts in contact with the lubricant and Lewis acids further increase lubricant degradation.

In order to improve the thermal stability of fluorinated oils, additives may be added; such additives are typically fluorinated polymers bearing at one or both ends of the polymer chain aromatic non fluorinated groups that may contain heteroatoms, such as nitrogen and phosphorus. However, the synthesis of these additives is in some instances troublesome; furthermore, certain non-fluorinated end groups are linked to the polymer chain through moieties like —CH₂OCH₂—, —CH₂(OCH₂CH₂)ₘO—, —C(O)O— and —C(O)N(R), which undergo degradation under severe conditions.

EP 1354932 A (SOLVAY SOLEXIS SPA) 22 Oct. 2003 discloses stabilizing additives having a (per)fluoropolyether chain with nitro-containing phenyl end groups; such end groups may be linked to the (per)fluoropolyether chain through a —CH₂O— moiety. However, these additives undergo phase separation upon storage at room temperature.

US 2007049502 (E.I. DU PONT DE NEMOURS AND COMPANY) 1 Mar. 2007 discloses compositions to be used as lubricants or lubricant additives which comprise mono- and/or bi-functional aryl perfluoropolyethers.

The monofunctional aryl perfluoropolyether complies with formula:

R_{f}—(Y)ₐ—(CₜR₍ᵤ₊ᵥ₎)—(O—CₜR¹₍ᵤ₊ᵥ₎)_{b}—R,

while the difunctional aryl perfluoropolyether complies with formula:

R_{f}¹—[(Y)ₐ—(CₜR₍ᵤ₊ᵥ₎)—(O—CₜR¹₍ᵤ₊ᵥ₎)_{b}—R]₂

wherein:

R_{f} and R_{f}¹ are respectively a monovalent or divalent straight or branched (per)fluoropolyether chain having formula weight from about 400 to 15,000:

Y is a divalent radical selected from a group which comprises, inter alia, —CH₂O— and —CF₂—;

a is 0 or 1;

(CₜR₍ᵤ₊ᵥ₎)ₜ is a divalent aryl group;

(O—CₜR¹₍ᵤ₊ᵥ₎)_{b} is a divalent aryloxy group;

R is selected from a group comprising hydrogen and nitro groups, but it cannot be solely hydrogen or nitro or combinations thereof;

each R¹ is selected from a group which comprises hydrogen and nitro groups;

t is equal to 6+u;

u is any combinations of 0, 2, 4, 6, 8, 10, 12, 14, 16;

v is independently 2 or 4;

b is 0-5.

All eighteen examples of this prior art document relate to the synthesis of monofunctional aryl derivatives of branched (per)fluoropolyethers comprising (OCF(CF₃)CF₂) units wherein an aryl group (namely a phenyl group) or an aryloxyaryl (namely a phenoxyphenyl) group is linked to the R_{f} chain via a —CF₂— moiety. Examples 7 and 8 relate to phenoxyphenyl derivatives and example 8 in particular discloses the preparation of a nitrodiphenyl ether derivative by nitration of the diphenylether derivative of example 7, whereby a nitro group is inserted on the phenoxy group.

EP 0528043 A (ASAHI CHEMICAL IND) 24 Feb. 1993 relates to a refrigerant composition comprising a fluoroalkane refrigerant and a fluoroaromatic lubricant which is compatible with the refrigerant and is said to be endowed with low moisture absorption properties and durability. The lubricant complies with formula (I):

R(XR_{f})ₙ

wherein:

X can be oxygen,

R is an n-valent unsubstituted or substituted aromatic group comprising at least one unsubstituted or substituted aromatic ring and having from 6 to 60 carbon atoms, n is an integer from 1 to 4 and R_{f} is a substituted or unsubstituted C₁-C₂₅ fluorocarbon chain which may comprise an oxygen atom in the main chain. Although nitro groups are mentioned among the possible substituents of group R, no nitro-substituted aromatic group is specifically disclosed. Furthermore, this prior art does not contain any hint or suggestion pointing at the selection of compounds wherein a nitro-substituted aromatic group is linked to a perfluoropolyether chain via a —CH₂O— or a —CF₂— group.

U.S. Pat. No. 5,104,559 (DOW CHEMICAL CO) 14 Apr. 1992 discloses lubricating compounds of formula:

R¹—R_{f}—O—Ar—R²

wherein:

R¹ is, inter alia, perfluoroalkoxy;

R_{f} is a perfluoroalkyl chain from 2 to 10 carbon atoms;

Ar is, inter alia, a biphenyl radical and R² is, inter alia, a nitro group.

The lubricants of formula R¹—R_{f}—O—Ar—R² can be manufactured according to a procedure comprising the base-catalysed addition of hydroxy-substituted aryl compounds to terminal perfluoroolefins. This prior art does not contain any hint or suggestion that would have prompted a skilled person to replace the perfluoroalkyl chain R_{f} with a (per)fluoropolyether chain and to select a biphenyl radical containing a nitro group.

U.S. Pat. No. 4,941,987 (AUSIMONT SPA) 17 Jul. 1990 discloses mono- or difunctional (per)fluoropolyether derivatives having an average molecular weight ranging from 1,500 to 10,000 to be used as lubricating greases which comply with formula:

R—O-Q-CFX—(Y)ₙ—Z

wherein:

R is a perfluoroalkyl radical or Z—(Y)ₙ—CFX—;

Q is a perfluoroalkyl polyether chain or a fluoroalkyl polyether chain;

X is fluorine or trifluoromethyl;

Y is a linking divalent radical which comprises —CH₂O— and —CF₂—;

n is 0 or 1;

and Z can be an aromatic radical containing 6 to 10 carbon atoms and optionally substituted with one or more nitro groups. The sole specifically disclosed compound wherein Z bears a nitro group are those of examples 1 and 3, in which Z is p-nitrophenyl.

US 2010261039 A (HOYA CORP [JP]) 14 Oct. 2010 discloses magnetic discs with a lubricant comprising a perfluoropolyether chain having an aromatic group at a chain end. Among aromatic groups, a biphenylene group is cited at par. [0048]. Nevertheless, this document does not specifically disclose diphenylene groups bearing nitro groups,

EP 1659165 A (SOLVAY SOLEXIS SPA) 24 May 2006 discloses PFPE pyridine derivatives used in the preparation of lubricant compositions; in particular, example 4 discloses a compound containing a PFPE chain having 2,4-dinitrophenyl end groups. This document does not disclose diphenyl or terphenyl groups as groups A.

WEBSTER, J. A., et al. Synthesis and Properties of Imide and Isocyanurate-Linked Fluorocarbon POlymers. Advances in Chemistry Series, Am. Chem. Soc. 1 Jan. 1973, no. 129, p. 61-79. discloses a perfluoropolyether derivative (specific reference is made to formula (IV) on page 65) comprising a perfluoropolyether chain having two chain ends, each end bearing a 2-nitrophenyl group, as intermediate for the preparation of isocyanatomethoxy derivatives used in the preparation of isocyanurate-linked hydrocarbon polymers.

EP 2135889 A (UNIMATEC CO LTD) 23 Dec. 2009 and EP 2280037 A (UNIMATEC CO LTD) 2 Feb. 2011 disclose PFPE derivatives having phenyl end groups linked to the PFPE chain through amido bridges, wherein the phenyl groups bear iodine and/or bromine atoms. Such derivatives are said to be curable and applicable to molding processes,

There is therefore the need of providing further compounds to be used as lubricants or additives for fluorinated oils or greases that are stable at very high temperatures and that can be manufactured in a convenient way.

### SUMMARY OF INVENTION

It has now been found that the above-mentioned need is met by the (per)fluoropolyether compounds according to the present invention, i.e. (per)fluoropolyether compounds comprising a (per)fluoropolyether chain (R_{f}) having at least two chain ends, wherein at least one of said chain ends have a bi- or ter-phenyl group bearing at least one nitro group and, optionally, one or more further substituents. Preferably, the (per)fluoropolyether compounds of the invention comprise a (per)fluoropolyether chain (R_{f}) having two chain ends, wherein at least one of said chain ends have a bi- or ter-phenyl group bearing at least one nitro group and, optionally, one or more further substituents.

Typically, the (per)fluoropolyether compounds according to the present invention comprise a fully or partially fluorinated polyoxyalkylene chain (R_{f}) comprising repeating units R°, statistically distributed along the chain, said repeating units being selected from the groups consisting of: —CFXO—, —CF₂ CF₂O—, —CF₂CF₂CF₂O—, —CF₂CF(CF₃)O—, —CF(CF₃)CF₂O—, —CF₂CF₂CF₂CF₂O—, —CR¹R²CF₂CF₂O— wherein R¹ and R², equal to or different from one another, represent hydrogen, chlorine, a (per)fluoroalkoxy group preferably containing from 1 to 12 carbon atoms or a (per)fluoroalkyl group preferably containing from 1 to 4 carbon atoms.

Chain R_{f} is preferably a straight (per)fluoropolyoxyalkylene chain complying with formula (A) below:

—(CF₂CF₂O)ₘ(CF₂O)ₙ(CF₂(CF₂)_{z}O)ₕ— (A)

in which m and n are integers and h is 0 or an integer, selected in such a way that the number average molecular weight of the chain ranges from 1,500 to 4,000, m/n is between 0.1 and 10; h/c+d is between 0 and 0.05 and z is 2 or 3.

In the following description, the term “ter-phenyl” is meant to comprise ortho-, metha- and para-terphenyl isomers; however, the para-isomer is preferred.

Preferably, the bi- or ter-phenyl group bears at least one nitro group at the ortho position. More preferably, the bi- or ter-phenyl group bears only one nitro group at the ortho position.

According to a preferred embodiment, the (per)fluoropolyethers of the invention are mono-functional, i.e. they comprise a (per)fluoropolyether chain (R_{f}) having two chain ends, wherein one chain end has a bi- or ter-phenyl group bearing at least one nitro group, preferably at least one nitro group at the ortho position.

According to another preferred embodiment, the (per)fluoropolyethers of the invention are bi-functional (per)fluoropolyethers, i.e. they comprise a (per)fluoropolyether chain (R_{f}) having two chain ends, wherein both chain ends have a bi- or ter-phenyl group bearing at least one nitro group, preferably at least one nitro group at the ortho position. Bifunctional (per)fluoropolyethers are particularly preferred.

Further to the at least one nitro group, the bi- or ter-phenyl group may optionally contain one or more substituents other than nitro (herein after also referred to as “further substituents”), for example one or more carboxy, secondary or tertiary amino, thio or phosphate groups.

For the sake of clarity, in the following description:

the prefix “(per)” indicates that the compound, chain, group or moiety following the prefix may be fully or partially fluorinated;

the acronym “PFPE” stands for (per)fluoropolyether;

the expression “ortho position” refers to the carbon atom of the bi-phenyl or ter-phenyl ring adjacent to the carbon atom of the phenyl ring linked to the R_{f} chain, possibly via a linking moiety, such as linking moiety Las defined further below;

unless otherwise indicated, the term “halogen” is intended to comprise fluorine, chlorine, bromine and iodine;

the above-indicated preferred embodiments, concerning the compounds of the invention, in particular those concerning the R_{f} chain, the number and position of the nitro group(s) apply also to the further preferred embodiments disclosed below.

The compounds of the invention preferably comply with general formula (I) below:

R—O—R_{f}—CFX-L-Ar (I)

wherein:

R is a (per)fluoroalkyl group containing from 1 to 3 carbon atoms or is Ar-L-CFX—;

R_{f} is a (per)fluoropolyoxyalkylene chain as defined above;

X is fluorine or —CF₃;

L is —CF₂— or —CH₂O—;

Ar is a bi- or ter-phenyl group bearing at least one nitro group and, optionally, one or more further substituents preferably selected among those indicated above.

In the compounds of formula (I) Ar is preferably a bi- or para ter-phenyl group bearing at least one nitro group at the ortho position. More preferably, Ar is a bi- or para ter-phenyl group bearing only one nitro group at the ortho position.

The compounds of general formula (I) in which L is —CH₂O—, herein after referred to as compounds (Ia) of formula:

R—O—R_{f}—CFX—CH₂O—Ar (Ia)

wherein:

R is a (per)fluoroalkyl group containing from 1 to 3 carbon atoms or Ar—OCH₂—CFX— and R_{f}, X and Ar are as defined above; can be manufactured by nucleophile reaction of a PFPE alcohol:

with a bi- or ter-phenyl compound bearing a leaving group and at least one nitro group and, optionally, one or more substituents as defined above or

with a phenyl or bi-phenyl compound bearing a leaving group and, optionally, one or more substituents as defined above, and subsequent reaction with a bi-phenyl or ter-phenyl compound, wherein at least one nitro group (and, optionally, one ore more substituents as defined above) is present on either the phenyl or bi-phenyl compound.

The compounds of formula (Ia) in which Ar is a biphenyl group of formula (IIa) below:

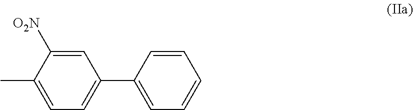 [SMILES: Cc1ccc(-c2ccccc2)cc1[N+](=O)[O-]]

or a terphenyl group of formula (IIb) below:

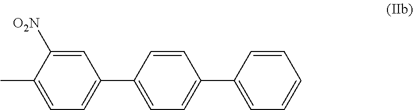 [SMILES: Cc1ccc(-c2ccc(-c3ccccc3)cc2)cc1[N+](=O)[O-]]

are preferred; bifunctional compounds, i.e. those in which R is Ar—OCH₂ —CFX—, wherein R is a group (IIa) or (IIb) are particularly preferred.

The compounds (Ia) in which Ar complies with the above formula (IIa) or (IIb) can be manufactured through a process [process (P1)] which comprises the following steps:

a) reaction of a (per)fluoropolyether of formula (III):

R—O—R_{f}—CFX—CH₂OH (III)

wherein R is a (per)fluoroalkyl group containing from 1 to 3 carbon atoms or HOCH₂—CFX— and R_{f} and X are as defined above;

with a compound of formula (IV):

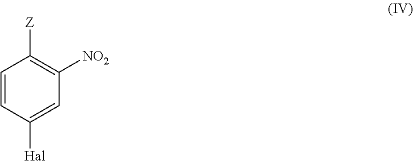

wherein Z is a leaving group typically selected from halogen, triflate and nonaflate and Hal is a halogen selected from fluorine, chlorine, bromine and iodine, with the proviso that, when Z is halogen, Hal is a halogen atom less reactive to nucleophile substitution;

to provide a compound of formula (V):

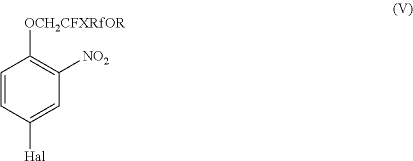

wherein X, R_{f} and Hal are as defined above and R is a (per)fluoroalkyl group containing from 1 to 3 carbon atoms or a group of formula (VI):

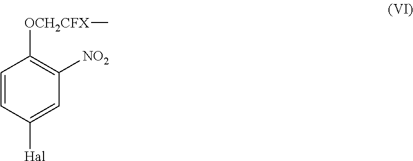

wherein X and Hal are as defined above;

b) Suzuki condensation of the compound of formula (VI) with benzene boronic or biphenyl boronic acid to provide a compound of formula (Ia) in which Ar is a group of formula (IIa) or (IIb).

Step a) may be carried out either in the absence or in the presence of a solvent. Typically, the reaction is carried out in the presence of polar or protic solvents, by reacting the (per)fluoropolyether (III) with the compound of formula (IV) at an equivalent ratio [i.e. the ratio between the reactive hydroxy group(s) of the PFPE of formula (III) and the leaving group Z on the compound of formula (IV)] usually ranging from 1 to 2 in the presence of a base, which can be selected from an organic or inorganic base. Suitable organic bases are, for example, tertiary amines like triethylamine and potassium ter-butoxide. Suitable inorganic bases are, for example, sodium and potassium hydroxide and sodium and potassium carbonate. According to a preferred embodiment, the compound of formula (IV) is 4-bromo-1-fluoro-nitrobenzene and the base is potassium ter-butoxide. Usually, the (per)fluoropolyether (III) and the compound (IV) are mixed together at room temperature, then the solvent (if used) and the base is(are) added and the temperature is increased up to values ranging from 30° C. to 100° C. until completion of the reaction. Compound (V) can be repeatedly washed with an organic solvent, e.g. n-hexane, toluene and benzene, in order to remove any unreacted compound (IV). If step a) is carried out in the presence of a solvent, such a solvent is removed before submitting compound (V) to the washing procedure.

Step b) can be carried out according to known methods for Suzuki reactions, for example those disclosed in MIYAURA, Norio, et al. A new stereospecific cross-coupling by the palladium-catalyzed reaction of 1-alkenylboranes with 1-alkenyl or 1-alkynyl halides. Tetrahedron Letters: Tetrahedron lett: 0040-4039. 1979, vol. 20, no. 36, p. 3437-3440. and MIYAURA, Norio, et al. Palladium-Catalysed Cross Coupling Reactions of Organoboron Compounds. Chemical Reviews. 1995, vol. 95, no. 7, p. 2457-2883. Typically, compound (V) is reacted with boronic acid or with biphenyl boronic acid in the presence of a palladium catalyst, preferably (PPh₃)₄Pd⁰ and of an inorganic base; a solvent is usually added in order to dissolve the base. Suitable inorganic bases are, for example, sodium, potassium and cesium carbonate, sodium bicarbonate and sodium and potassium phosphate, while suitable solvents are, for example, dimethylformamide, toluene, ethanol and tetrahydrofurane/water or dimethoxyethane/water mixtures. The reaction is typically carried out at a temperature ranging from 40 to 80° C. Upon completion of the reaction, the desired compound of formula (Ia) is recovered from the reaction mixture and optionally purified by chromatography.

It will be apparent to those skilled in the art that process (P1) can also be carried out using a compound of formula (IV) bearing one or more further nitro groups and/or further substituents as defined above and/or a benzene boronic or biphenyl boronic acid bearing one or more nitro groups and/or one or more further substituents as defined above.

The compounds (I) wherein L is a group of formula —CF₂—, herein after referred to as compounds (Ib) of formula:

R—O—R_{f}—CFX—CF₂—Ar (Ib)

in which R_{f} and X are as defined above, R is a (per)fluoroalkyl group containing from 1 to 3 carbon atoms or a group of formula Ar—CF₂—CFX— and Ar is a group of formula (IIa) or (IIb) as defined above are also preferred. Particularly preferred are bifunctional compounds, i.e. those in which R is Ar—CF₂—CFX—, wherein Ar is a group of formula (IIa) or (IIb). Compounds (Ib) in which Ar is a group of formula (IIa) or (IIb) can be manufactured by means of a process [process (P2)] which comprises the following steps:

a′) providing a compound of formula (VII):

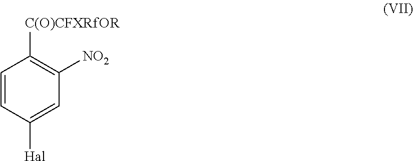

in which Rf, X and Hal are as defined above and R is a (per)fluoroalkyl group containing from 1 to 3 carbon atoms or a group of formula (VIII)

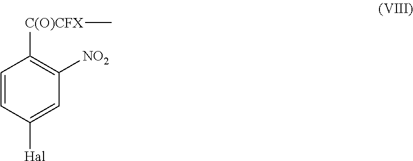

in which X and Hal are as defined above;

b′) Suzuki condensation of the compound of formula (VII) with benzene boronic or biphenyl boronic acid to provide a compound of formula (IX):

R—O—R_{f}—CFX—C(O)—Ar (IX)

in which R_{f} and X are as defined above and R is a (per)fluoroalkyl group containing from 1 to 3 carbon atoms or a group of formula (X):

Ar—C(O)—CFX— (X)

in which Ar is a group of formula (IIa) or (IIb);

c′) fluorination of the compound of formula (X) to provide a compound of formula (Ib) in which Ar is a group of formula (IIa) or (IIb).

The compounds of formula (VII) can be manufactured by reaction of (per)fluoropolyethers of formula (X):

R²—O—R_{f}—CFX—C(O)OR¹ (X)

in which R_{f} and X are as defined above, R¹ is lower alkyl, preferably methyl or ethyl, and R² is a (per)fluoroalkyl group containing from 1 to 3 carbon atoms or R¹0C(O)—CFX— with a compound of formula (IV) as defined above according to known methods for the synthesis of aromatic ketones, for examples as disclosed in PACIOREK, K J L, et al. Perfluoroalkyl- and perfluoroalkylether-substituted aromatic phosphates and phosphonates. Journal of Fluorine Chemistry. 1998, vol. 88, p. 55-61.

The compounds of formula (X) can be manufactured as disclosed, for example, in TONELLI, Claudio, et al. Linear perfluoropolyether difunctional oligomers: chemistry, properties and applications. Journal of Fluorine Chemistry. 1995, vol. 95, no. 1-2, p. 51-70. and in TONELLI, Claudio, et al. Perfluoropolyether alkyl diesters: Structure effects of the alkyl group on the kinetics of the hydrolysis reactions. Journal of Polymer Science—Part A—Polymer chemistry. 2002, vol. 40, no. 23, p. 4266-4280.

Step b′) can also be carried out according to known methods for Suzuki reactions as indicated for step b) above.

Step c′) can be carried out according to known methods for the fluorination of ketone compounds, for example as taught in SMITH, W. C., et al. Fluorination reaction of sulfur tetrafluoride. Journal of American Chemical Society. 1959, vol. 81, no. 12, p. 3165-3166. or in DINOIU, Vasile, et al. Chemical Fluorination of Organic Compounds. Revue Roumaine de Chimie. 2007, vol. 52, no. 3, p. 219-234. or in ROSSI, Francesco, et al. Process Research and Development and Scale-up of a 4,4-Difluoro-3,3-dimethylproline Derivative. Organic Process Research. 2008, vol. 12, no. 2, p. 322-338. According to a preferred embodiment, the fluorination reaction is carried out with SF₄ as fluorinating agent. Typically, the ketone compound of formula (IX) obtained in step b′) is dissolved in a suitable solvent or solvent mixture and added with SF₄ in a molar ratio (with respect to SF₄) ranging from 1.0 to 0.1. The reaction mass is heated up to a temperature ranging from 50° C. to 150° C. until completion of the reaction. The desired compound (Ib) is then isolated from the reaction mass and optionally purified by chromatography.

It will be apparent to those skilled in the art that the above illustrated process (P2) can also be carried out using a compound of formula (VII) in which the phenyl ring bears one or more further nitro groups and/or one or more further substituents and/or with a benzene boronic or biphenyl boronic acid bearing one or more nitro groups and/or one or more further substituents as defined above.

The compounds of the present invention are endowed with lubricant properties and are stable at very high temperatures; therefore, they can be conveniently used as base oils or as thermal stabilizers for the preparation of lubricant compositions in the form or oils or greases.

Thus, a further object of the present invention is represented by lubricant compositions in the form of greases or oils comprising one or more compounds according to the present invention.

It is generally understood that oils are compounds having kinematic viscosity (ASTM D445) at 40° C. of from 30 to 30 000 cSt; greases are derived from such oils by addition of suitable thickeners, such as notably polytetrafluoroethylene (PTFE) or inorganic compounds, e.g. talc.

Generally, the lubricant compositions according to the present invention will have a kinematic viscosity in the above mentioned conditions of 30 to 3,000 cSt, preferably from 50 to 500 cSt, when determined at 20° C. according to ASTM D445.

According to a first preferred embodiment, lubricant compositions in the form of oils [hereinafter compositions (C1)] containing the compounds of the invention as thermal stabilizers comprise, preferably consist of:

(i) one or more (per)fluoropolyether compounds according to the invention, preferably in an amount ranging from 0.1% to 10% wt;

(ii) one or more base oils, preferably in an amount ranging from 90% to 99% wt;

(iii) optionally, one or more thickening agents, preferably in an amount ranging from 10% to 50% wt and

(iv) optionally, one or more additives, preferably in an amount ranging from 1% to 5% wt.

Preferred compounds according to the invention to be used as thermal stabilizers in compositions (C1) are those complying with formulae (Ia) and (Ib) above.

Base oils may be fluorinated or non fluorinated (hydrogenated) oils or mixtures thereof. Preferably, fluorinated oils are (per)fluoropolyether oils; examples of (per)fluoropolyether oils (iii) suitable for the preparation of the compositions (C1) are those identified as formulae (Ia)-(8a) in EP 2089443 A (SOLVAY SOLEXIS SPA) or as formulae (1)-(8) in WO 2009/141284 (SOLVAY SOLEXIS SPA) (SOLVAY SOLEXIS SPA) or as formulae (1) to (9) in WO 2011/042374 (SOLVAY SOLEXIS SPA). More preferably, the (per)fluoropolyether oils are those commercially available under the trade name FOMBLIN® (type Y, M, W, or Z) from Solvay Specialty Polymers S.p.A.; such oils generally comprise at least one oil (i.e. only one or mixture of more than one oil) complying with either of formulae here below:

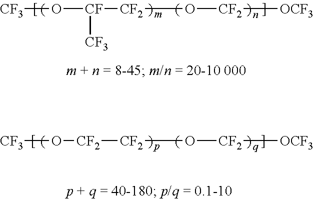

One or more compounds according to the present invention different from the one using as ingredient (i) can also be used as fluorinated base oil (ii) in compositions (C1); for example, if a compound (Ia) is used as ingredient (i), one or more compound(s) (Ia) different from the one used as ingredient (i) or one or more compounds (Ib) can be used as ingredient (ii).

Suitable non fluorinated oils to be used as base oils in compositions (C1) are preferably selected from mineral, paraffinic, aromatic oils, polyalphaolefins, alkyl esters, silicone esters, naphthalene derivatives, polyalkylated cycloalkanes, polyphenylethers.

According to a second preferred embodiment, lubricant compositions in the form of greases [herein after compositions (C2)] containing the compounds of the invention comprise, preferably consists of:

(i*) a (per)fluoropolyether compound according to the invention, preferably in an amount ranging from 50% to 90% wt;

(iii*) one or more thickening agent, preferably in an amount ranging from 10% to 50% wt and;

(iv*) optionally, one or more additives.

Preferred (per)fluoropolyether compounds of the invention to be used as as ingredient (i*) in compositions (C2) are those complying with formula (Ia) above.

Examples of suitable thickening agents are PTFE, talc, silica, boron nitride, polyurea, alkali or alkali-earth metals terephthalate, calcium and lithium soaps and complexes thereof; among them, PTFE is preferred.

Examples of suitable additives are antirust agents, antioxidants, thermal stabilizers, pour point depressants, antiwear agents, including those for high pressures, dispersants, tracers, dyestuffs, talc and inorganic fillers. Examples of dispersants are, for example, surfactants, preferably non-ionic surfactants, more preferably (per)fluoropolyether surfactants and (per)fluoroalkyl surfactants. Examples anti-wear additives are phosphazene derivatives of (per)fluoropolyethers, like those disclosed in EP 1336614 A (SOLVAY SOLEXIS SPA).

The invention will be disclosed in greater detail by means of the following experimental section.

Should the disclosure of any patents, patent applications, and publications which are incorporated herein by reference conflict with the description of the present application to the extent that it may render a term unclear, the present description shall take precedence.

Experimental Section

Materials and Methods

The (per)fluoropolyether diols used as reagents in examples 1-4 were prepared following the procedure disclosed in U.S. Pat. No. 3,847,978 (MONTEDISON SPA) Dec. 11, 1974, in BANKS, R. E., et al. Organofluorine Chemistry: Principles and Commercial Applications. New York: Plenum Press, 1994.

The (per)fluoropolyether diesters used as reagents in examples 10-13 were prepared following the procedure disclosed in TONELLI, Claudio, et al. Linear perfluoropolyether difunctional oligomers: chemistry, properties and applications. Journal of Fluorine Chemistry. 1995, vol. 95, no. 1-2, p. 51-70. and in TONELLI, Claudio, et al. Perfluoropolyether alkyl diesters:Structure effects of the alkyl group on the kinetics of the hydrolysis reactions. Journal of Polymer Science—Part A—Polymer chemistry. 2002, vol. 40, no. 23, p. 4266-4280.

The rest of the reagents and solvents were commercially available and were used without further purification.

The procedure for the thermooxidation test and for the tribological test are described below.

In the following, Mₙ identifies the average numerical weight of chain R_{f} determined by ¹⁹F-NMR analysis.

### EXAMPLES 1-4

### Synthesis of Compounds (V) Having Formula (V₁)

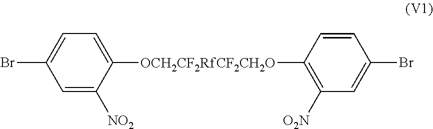

### EXAMPLE 1

### Synthesis of a Compound (V₁) Wherein Rf=(OCF₂CF₂O)_{m (CF}₂O)ₙ; m/n=1; Mn=1500

160 g (214 meq) HOCH₂CF₂(OCF₂CF₂O)ₘ(CF₂O)ₙCF₂CH₂OH wherein m/n=1 and m+n selected in such a way that Mₙ was 1500 was placed vacuum dried in a 2 L, four-necked flat bottomed cylindrical flask equipped with a overhead stirrer, a septum adaptor, a thermowell and a dropping funnel.

55 g (250 meq) 4-bromo-1-fluoro-2-nitrobenzene was subsequently added via a cannula, followed by the addition of ter-BuOH (1200 ml). The mixture was stirred at room temperature for 3 min. Potassium ter-butoxide (250 meq) dissolved in t-BuOH (400 ml) was then added to the reaction mixture via a cannula over a period of 60 min. The reaction mixture was heated at 80° C. for 6 h, ter-BuOH was removed under reduced pressure and the reaction mixture was repeatedly washed with water until neutrality. Finally, the reaction mass was repeatedly washed with n-hexane until complete removal of excess 4-bromo-1-fluoro-2-nitrobenzene (as confirmed by TLC analysis).

190 g of the desired compound (V¹) was isolated after drying at 50° C. under reduced pressure for 8 h (structure confirmed by ¹⁹F-NMR and ¹H-NMR analyses.

### EXAMPLE 2

### Synthesis of a Compound (V₁) Wherein Rf=(OCF₂CF₂O)ₘ (CF₂O)ₙ; m/n=1; Mn=2000

Following the procedure of example 1, 180 g of PFPE diol of formula:

HOCH₂CF₂(OCF₂CF₂O)ₘ(CF₂O)ₙCF₂CH₂OH

wherein m/n=1 and m+n were selected in such a way that the Mₙ was 2000 were reacted with 4-bromo-1-fluoro-2-nitrobenzene.

The reaction product was also worked up as described in the example 1.

207 g of the desired compound (V¹) were obtained (structure confirmed by ¹⁹F-NMR and ¹H-NMR analyses).

### EXAMPLE 3

### Synthesis of Compound (V₁) wherein Rf=(OCF₂CF₂O)ₘ(CF₂O)ₙ; m/n=1; Mn=3000

250 g of PFPE diol of formula:

HOCH₂CF₂(OCF₂CF₂O)ₘ(CF₂O)ₙCF₂CH₂OH

wherein m/n=1 and m+n were selected in such a way that Mₙ was 3000 were reacted with 4-bromo-1-fluoro-2-nitrobenzene.

The reaction product was also worked up as described in example 1.

280 g of the desired compound (V¹) were obtained (structure confirmed by ¹⁹F-NMR and ¹H-NMR analyses).

### EXAMPLE 4

### Synthesis of a Compound (V₁) wherein Rf=(OCF₂CF₂O)ₘ(CF₂O)ₙ; m/n=1; Mn=4000

250 g of PFPE diol of formula:

HOCH₂CF₂(OCF₂CF₂O)ₘ(CF₂O)ₙCF₂CH₂OH

wherein m/n=1 and m+n selected in such a way that the Mₙ was 4000 were reacted with 4-bromo-1-fluoro-2-nitrobenzene.

The reaction product was also worked up as described in example 1.

285 g of the desired compound (V¹) were obtained.

### EXAMPLES 5-8

### Synthesis of a Biphenyl Compound (Ia) of Formula (Ia₁)

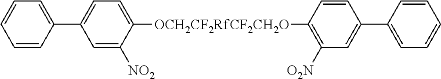

### EXAMPLE 5

### Synthesis of Compound (Ia₁) wherein Rf=(OCF₂CF₂O)ₘ(CF₂O)ₙ; m/n=1; Mn=1500

60 g (63 meq) of the compound (V¹) obtained according to example 1 were placed in a 2 L four-necked cylindrical flat bottom flask, equipped with a overhead stirrer and a septum adaptor. Thereafter, 11.5 g (91 meq) phenylboronic acid were added, together with 13.4 g (121 meq) sodium carbonate and 0.9 g (PPh₃)₄Pd(0) as catalyst. The reaction mass was degassed by applying vacuum for a few minutes, followed by refilling with nitrogen gas. 1.2 L THF and 150 ml distilled water (to dissolve sodium carbonate) were added to the mixture using a syringe. The reaction mixture was stirred at 70° C. The reaction mass initially turned yellow and gradually turned dark-brown over a period of 30 min. The reaction was continued at 70° C. for 5 h. THF was removed under reduced pressure and the reaction mixture was washed with water (5×200 ml). Then the reaction mass was taken up in ethyl acetate (400 ml) and the solution dried over sodium sulphate. Ethyl acetate was removed under reduced pressure and the product was purified by column chromatography on silica gel using petroleum ether:ethyl acetate (80:20 v/v) as eluent.

54 g of the desired compound (Ia¹) were isolated (structure confirmed by ¹⁹F-NMR and ¹H-NMR analyses).

### EXAMPLE 6

### Synthesis of Compound (Ia₁) wherein Rf=(OCF₂CF₂O)ₘ(CF₂O)ₙ; m/n=1; Mn=2000

70 g of the compound (V¹) prepared according to example 2 were converted into the corresponding compound (Ia) following the procedure of example 5.

65 g of the desired compound (Ia¹) was isolated (structure confirmed by ¹⁹F-NMR and ¹H-NMR analyses).

### EXAMPLE 7

### Synthesis of Compound (Ia₁) wherein Rf=(OCF₂CF₂O)ₘ(CF₂O)ₙ; m/n=1; Mn=3000

80 g of compound (V¹) prepared according to example 3 were converted into the corresponding compound (Ia¹) following the procedure of example 5.

73 g of the desired compound (Ia¹) was isolated (structure confirmed by ¹⁹F-NMR and ¹H-NMR analyses).

### EXAMPLE 8

### Synthesis of Compound (Ia₁) wherein Rf=(OCF₂CF₂O)ₘ(CF₂O)ₙ; m/n=1; Mn=4000

85 g of compound (V¹) prepared according to example 4 were converted into the corresponding compound (Ia¹) following the procedure of example 5.

78 g of the desired compound (Ia¹) was isolated, as confirmed by ¹⁹F-NMR and ¹H-NMR.

### EXAMPLE 9

### Synthesis of a Terphenyl Compound (Ia) of Formula

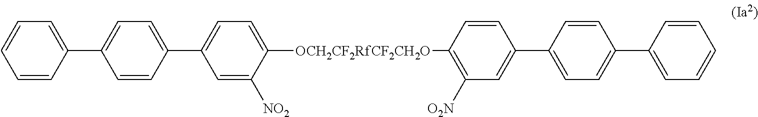 [0158] wherein Rf=(OCF₂CF₂O)ₘ(CF₂O)ₙ; m/n=1; Mn=2000

10 g of the compound (V¹) prepared according to example 2 were converted into the corresponding ter-phenyl derivative following the procedure of example 5 with the difference that biphenylboronic acid was used instead of phenyl boronic acid; all molar ratio among reagents were unchanged.

8 g of the desired terphenyl compound (Ia²) were obtained (structure confirmed by ¹⁹F-NMR and ¹H-NMR analyses).

### EXAMPLES 10-13

### Synthesis of a Diphenyl Derivative (Ib) of Formula (Ib₁)

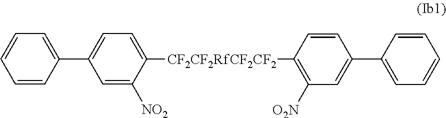

### EXAMPLE 10

### Synthesis of Diphenyl Derivative (Ib₁) Wherein Rf=(OCF₂CF₂O)ₘ(CF₂O)ₙ; m/n=1; Mn=1500

Step a)—Synthesis of a Compound (VII) of Formula (VII₁):

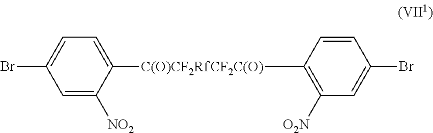

To a solution of 42 g (150 mmoles) of 1,4-dibromo-2-nitrobenzene in 500 ml diethyl ether at −78° C., 62 ml of n-butyllithium (2.42 M in n-hexane), corresponding to 150 mmoles, was added over a period of about 2 hrs. The solution turned from colourless to a cloudy pale yellow and the temperature raised few degrees. The reaction mass was cooled down again to −78° C., then it was added to 106.6 g (142 meq) PFPE diethyl ester of formula:

EtO(O)CCF₂(OCF₂CF₂O)ₘ(CF₂O)ₙCF₂C(O)O Et

wherein m/n=2 and m+n selected in such a way that the average numerical molecular weight of the PFPE chain was 1500 over a period of 2 h.

A pale yellow reaction mass was obtained, which was maintained under stirring for 1 extra hr at −75/−78° C. After this time, the mixture was hydrolyzed with 500 ml HCl (2N).

The hydrolyzed mixture was allowed to warm up to room temperature, then the organic phase was separated. The aqueous layer was extracted twice with a mixture of CFC113/ethyl ether (50:50 v/v), in order to recover any compound (VII¹) possibly dissolved therein. The two extraction mixtures were pooled together and added to the organic phase, then the low boiling solvents were gently removed by distillation under reduced pressure, thereby obtaining 100 g (VII¹) (structure confirmed by ¹⁹F-NMR and ¹H-NMR analyses).

Step b) Synthesis of a Compound (IX) of Formula (IX₁):

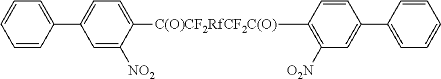

30 g of the compound (VII¹) obtained in step a) was converted into the corresponding bi-phenyl (IX¹) by Suzuki reaction, following the procedure of example 5 above.

27 g of compound (IX¹) was isolated, as confirmed by ¹⁹F-NMR and ¹H-NMR analyses.

Step c) Fluorination of Compound (IX₁)

50 g (53 meq) of compound (IX¹) obtained in step b) were placed into a 250 ml Hastelloy C pressure vessel. The equipment was cooled in a dry ice-acetone bath, then about 15 ml anhydrous HF, about 22 g SF₄ and 70 ml FCF 113 were added. The inlet was closed and the vessel was warmed up to 110° C. for 48 h. After this time, the vessel was cooled, vented, and the liquid reaction mass was treated with about 20 g KF. The reaction mass was filtered, the liquid phase was recovered and the solvent was evaporated off, then volatile by-products were removed by distillation under reduced pressure, and a final thin layer fractionation made it possible to isolate, as distilled fraction, the desired compound (Ib¹) (39 g, 41 meq) (structure confirmed by IR, ¹⁹F-NMR and ¹H-NMR analyses).

### EXAMPLE 11

### Synthesis of Diphenyl Derivative (Ib₁) Wherein Rf=(OCF₂CF₂O)ₘ(CF₂O)ₙ; m/n=1; Mn of Rf=2000

The procedure of example 10 was followed, using as reagent the PFPE diethyl ester of formula:

EtO(O)CCF₂(OCF₂CF₂O)ₘ(CF₂O)ₙCF₂C(O)O Et

wherein m/n=2 and m+n selected in such a way that the average numerical molecular weight of the (per)fluoropolyether chain was 2000.

40 g of the desired compound (Ib¹) was obtained (structure confirmed by IR, ¹⁹F NMR and ¹H NMR analyses).

### EXAMPLE 12

### Synthesis of Diphenyl Derivative (Ib₁) Wherein Rf=(OCF₂CF₂O)ₘ(CF₂O)ₙ; m/n=1; Mn 3000

The procedure of example 10 was followed, using as reagent the PFPE diethyl ester of formula:

EtO(O)CCF₂(OCF₂CF₂O)ₘ(CF₂O)ₙCF₂C(O)O Et

wherein m/n=2 and m+n selected in such a way that the average numerical molecular weight was 3000.

30 g of the desired compound (Ib¹) were obtained (structure confirmed by IR, ¹⁹F-NMR and ¹H-NMR analyses).

### EXAMPLE 13

### Synthesis of the Above Diphenyl Derivative (Ib) Wherein Rf=(OCF₂CF₂O)ₘ(CF₂O)ₙ; m/n=1; Mn=4000

The procedure of example 10 was followed using as reagent the PFPE diethyl ester of formula:

EtO(O)CCF₂(OCF₂CF₂O)ₘ(CF₂O)ₙCF₂C(O)O Et

wherein m/n=2 and m+n selected in such a way that the average numerical molecular weight of the PFPE chain was 4000

35 g of the desired compound (Ib¹) were obtained (structure confirmed by IR, ¹⁹F-NMR and ¹H-NMR analyses).

Tests

The compounds of examples 4 to 8 and 10 to 13 were tested as additives for imparting thermoxidative stability to PFPE fluids and greases.

The compounds of examples from 5 to 8 were also evaluated as such as lubricants under severe conditions (see tribological test below).

Test 1—Thermooxidation Test

The thermooxidation test was carried out using the equipment described in: SNYDER, Carl E., et al. Development of Polyperfluoroalkylethers as High Temperature Lubricants and Hydraulic Fluids. ASLE Transactions. 1975, vol. 3, no. 13, p. 171-180. The operating conditions were as follows:
Test temperature: 270° C. for oils and 250° C. for greases;
Air flow: 1 L/h;
Metals dipped in the fluid: stainless steel (AISI 304) and Ti alloy (Al 6%, V 4%).

A sample of the fluid to be tested, containing the compounds of examples 5 to 8 and 10 to 14 as additives in the amount specifically indicated (typically between 0.5-5% w/w), was introduced in the glass test tube of the equipment (see for example FIG. 1 of the above-cited reference), the glass tube was weighted and heated at the test temperature. When the required time had elapsed, the glass test tube was cooled to room temperature and weighted again. The difference of the weight before and after heating, referred to the weight of the sample before the test, gave the percent weight loss of the tested fluid. At the end of the test the appearance of the metals dipped into the fluid was visually evaluated.

All tested formulations contained 1% w/w of the compounds (Ia) and (Ib)) of examples 5-8 and 10-13 according to the present invention in a Fomblin® M30 PFPE oil (Solvay Specialty Polymers), characterized by the following structure:

CF₃(OCF₂CF₂)ₙ(OCF₂)ₘOCF₃ (with m+n selected in such a way that the average numerical molecular weight Mₙ=9800)

The results of the tests are summarized in table 1 below, in which

Reference 1 is a Fomblin® M30 PFPE oil without additive.

TABLE 1

| | 24 h | 48 h | 72 h | 96 h | 192 h | 240 h |
|---|---|---|---|---|---|---|
| Product | Δw (%) | Δw (%) | Δw (%) | Δw (%) | Δw (%) | Δw (%) |
| Example 5 | 0.20 | 0.40 | 0.48 | 0.55 | 1.20 | 1.48 |
| Example 6 | 0.18 | 0.37 | 0.44 | 0.50 | 1.14 | 1.38 |
| Example 7 | 0.11 | 0.36 | 0.40 | 0.44 | 0.93 | 1.00 |
| Example 8 | 0.10 | 0.33 | 0.34 | 0.40 | 0.8 | 0.97 |
| Example 10 | 0.17 | 0.35 | 0.45 | 0.51 | 1.15 | 1.41 |
| Example 11 | 0.15 | 0.34 | 0.43 | 0.49 | 1.18 | 1.40 |
| Example 12 | 0.10 | 0.28 | 0.38 | 0.40 | 0.80 | 0.91 |
| Example 13 | 0.08 | 0.28 | 0.37 | 0.39 | 0.78 | 0.88 |
| Reference 1 | 45 | 80 | 100 | | | |

It stems from the above results that the even small amounts of compounds of the invention are able to increase thermooxidative stability of the Fomblin® M30 PFPE oil.

Two samples of greases were also prepared using 30% by weight of PTFE and 70% by weight of base oil Fomblin® PFPE M30. The first sample (sample 1) contained only PTFE and Fomblin® PFPE M30, while the second one (sample 2) was also added with 2% of the compound (Ia) of example 7; then 10% by weight of fine powdered iron was added to each sample. The penetration value of both greases, measured by ASTM D217 method, was 292 mm/10′.

50 g of these samples were then poured into a glass cup with an internal diameter of 96 mm and placed in a ventilated oven at 250° C. for 100 h. The weight loss of each cup was checked during the test to evaluate the stability of the greases. After 100 h the penetration of the greases was determined. The greases behaviour in terms of weight loss % as a function of time is summarized in the following table:

TABLE 2

| | | Additive | | Weight | | |
|---|---|---|---|---|---|---|
| | | (%) | | loss | | Penetra- |
| | | Product | Iron | 250° C. | Penetration | tion |
| Sample | Composition | 3 | powder | 100 h | t = 0 | t = 100 h |
| 1 | 70% M30 | — | 10% w | 54% | 292 | n.d.* |
| 2 | 30% PTFE | 2 | | 3% | | 289 |

*Not determined; sample to dry

The results reported in the above table confirm the very high efficiency of the compounds of the invention as thermal stabilizers for (per)fluorinated greases in air and in presence of metals.

Test 2—Tribological Test

The tribological properties of the compounds (Ia) of examples 5-8 were tested using a SRV® Test Machine. Their behavior was compared to that of a reference commercial product Fomblin® Z PFPE Tetraol 2000S (Solvay Specialty Polymers).

The ball-on-disk geometry was used.

This method allows the measure of the relationship between the load that the lubricant undergoes and the coefficient of friction (CoF) under a high frequency regime and with a linear oscillation.

The following experimental conditions were applied:

Temperature: 50° C.;

Frequency: 50 Hz;

Amplitude of the frequency (stroke): 1 mm;

Rate of the oscillation (sliding velocity): 0.1 m/s;

Initial load: 50 N for 2′, increment of 50 N for 2′, then increments of 100 N for 2′ up to the desired value.

The data in the table 3 below report the coefficient of friction (CoF) defined as the ratio between the parallel force to the oscillation and the normal force to the oscillation plane as a function of the applied load.

It stems from such data that the CoFs of the compounds (Ia) of examples 5 to 9 according to the invention are all better (i.e. lower) than that of Fomblin® Z PFPE Tetraol 2000S. Namely, an average value of CoF 0.08871 for the compounds of examples 5 to 8 vs a CoF value of 0.13401 for Fomblin® Z PFPE Tetraol 2000S was observed.

TABLE 3

| | Average CoF | | | | |
|---|---|---|---|---|---|
| | | | | Compound | Compound |
| | | Compound | Compound | of | of |
| Load | Reference | of | of | example | example |
| (N) | 2 | example 5 | example 6 | 7 | 8 |
| 50 | 0.13896 | 0.08927 | 0.08781 | 0.08935 | 0.09314 |
| 100 | 0.13401 | 0.08802 | 0.08828 | 0.08905 | 0.08950 |
| 200 | 0.12923 | 0.08708 | 0.09306 | 0.09385 | 0.09007 |
| 300 | 0.12758 | 0.09339 | 0.09507 | 0.09293 | 0.08509 |
| 400 | 0.12636 | 0.09306 | 0.12462 | 0.09969 | 0.17934 |

The compound of example 5 was tested in comparison with Reference 2 also at loads higher than 400 N; lower CoF values were observed in all tests, as is evident from table 4 below.

TABLE 4

| | Average CoF | |
|---|---|---|
| Load | Reference 2 | Compound of example 5 |
| 500 | 0.12509 | 0.09131 |
| 600 | 0.12567 | 0.08870 |
| 700 | 0.11382 | 0.08590 |
| 800 | 0.11175 | 0.09109 |

Test 3—Comparative Test

In this comparative test weight loss of two compounds according to the present invention was evaluated and compared with that of a compound according to prior art document EP 1354932.

The two compounds according to the present invention complied with general formula (Ia¹) above and had a molecular weight of 2200 and 3200 respectively, while the compound according to EP 1354932 complied with formula:

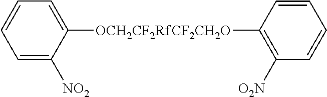

wherein Rf is as defined above and having a molecular weight of 2700.

Weight loss was evaluated by thermogravimetric analysis (TGA) under isothermal conditions in air at 250° C. for one hour. The results are reported in table 5 below.

TABLE 5

| Compound | Molecular weight | Weight loss (% w/w) |
|---|---|---|
| Formula (Ia¹) | 2200 | 0.60 |
| Formula (Ia¹) | 3200 | 0.40 |
| Compound according to | 2700 | 1.85 |
| EP 1354932 | | |

The results show that the compounds according to the present invention undergo a significantly lower weight loss than the prior art compound; it is pointed out that lower weight loss is observed also for the compound of formula (Ia¹) having a molecular weight lower than that of the prior art compound (2200 vs 2700). Therefore, the compounds of the invention can be used for the preparation of lubricant oils or greases having a constant composition over time, even under severe working conditions, e.g. temperature equal to or higher than 250° C.

1. A (per)fluoropolyether compound comprising a (per)fluoropolyether chain (R_{f}) having at least two chain ends, wherein at least one of said chain ends have a bi- or ter-phenyl group bearing at least one nitro group and, optionally, one or more further substituents.

2. A (per)fluoropolyether compound according to claim 1, wherein the (per)fluoropolyether chain (R_{f}) has two chain ends, and wherein one or both chain ends have a bi- or ter-phenyl group bearing at least one nitro group and, optionally, one or more further substituents.

3. The (per)fluoropolyether according to claim 1, wherein the (per)fluoropolyether chain (R_{f}) is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O— wherein R¹ and R², equal to or different from one another, represent hydrogen, chlorine, a (per)fluoroalkoxy group or a (per)fluoroalkyl group.

4. The (per)fluoropolyether compound according to claim 3, wherein R_{f} is a straight (per)fluoropolyoxyalkylene chain complying with formula (A):
—(CF₂CF₂O)ₘ(CF₂O)ₙ(CF₂(CF₂)_{z}O)ₕ— (A)
wherein m and n are integers and h is 0 or an integer, selected in such a way that the number average molecular weight of the chain ranges from 1,500 to 4,000, m/n is between 0.1 and 10; h/c+d is between 0 and 0.05 and z is 2 or 3.

5. The (per)fluoropolyether compound according to claim 1, wherein the bi- or ter-phenyl groups are bi- or ter-phenyl groups bearing at least one nitro group at the ortho-position and, optionally, one or more further substituents.

6. The (per)fluoropolyether compound according to claim 1, wherein the (per)fluoropolyether compound complies with formula (I):
R—O—R_{f}—CFX-L-Ar (I)
wherein:
R is a (per)fluoroalkyl group containing from 1 to 3 carbon atoms or is Ar-L-CFX—;
R_{f} is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O—0 wherein R¹ and R², equal to or different from one another, represent hydrogen, chlorine, a (per)fluoroalkoxy group or a (per)fluoroalkyl group;
X is fluorine or —CF₃;
L is —CF₂— or —CH₂O—;
Ar is a bi- or ter-phenyl group bearing at least one nitro group and, optionally, one or more further substituents.

7. The (per)fluoropolyether compound according to claim 6, wherein the (per)fluoropolyether compound complies with formula (Ia):
R—O—R_{f}—CFX—CH₂O—Ar (Ia)
wherein:
R is a (per)fluoroalkyl group containing from 1 to 3 carbon atoms or a group of formula Ar—OCH₂—CFX— wherein Ar is selected from:
a biphenyl group of formula (IIa): 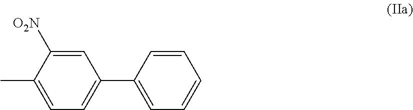 [SMILES: Cc1ccc(-c2ccccc2)cc1[N+](=O)[O-]]
and a terphenyl group of formula (IIb):

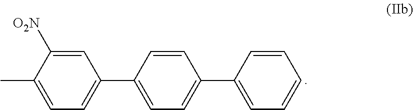 [SMILES: Cc1ccc(-c2ccc(-c3ccccc3)cc2)cc1[N+](=O)[O-]]

8. The (per)fluoropolyether compound according to claim 6, wherein the (per)fluoropolyether compound complies with formula (Ib):
R—O—R_{f}—CFX—CF₂—Ar (Ib)
wherein:
R is a (per)fluoroalkyl group containing from 1 to 3 carbon atoms or a group of formula Ar—CF₂—CFX— wherein Ar is selected from:
a biphenyl group of formula (IIa): 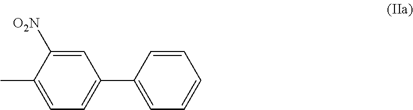 [SMILES: Cc1ccc(-c2ccccc2)cc1[N+](=O)[O-]]
and a terphenyl group of formula (IIb):

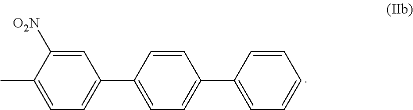 [SMILES: Cc1ccc(-c2ccc(-c3ccccc3)cc2)cc1[N+](=O)[O-]]

9. The (per)fluoropolyether compound according to claim 6, wherein R is a group of formula Ar-L-CFX.

10. A process for the manufacture of the (per)fluoropolyether compounds of claim 7, the process comprising:
reacting a (per)fluoropolyether compound of formula (III):
R—O—R_{f}—CFX—CH₂OH (III)
wherein R is a (per)fluoroalkyl group containing from 1 to 3 carbon atoms or HOCH₂—CFX—;
R_{f} is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O— wherein R¹ and R², equal to or different from one another, represent hydrogen, chlorine, a (per)fluoroalkoxy group or a (per)fluoroalkyl group; and
X is fluorine or —CF₃;
with a compound of formula (IV): 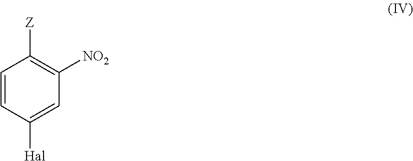
wherein Z is a leaving group and Hal is a halogen selected from fluorine, chlorine, bromine and iodine, with the proviso that, when the leaving group Z is halogen, Hal is a halogen atom less reactive to nucleophile substitution;
to provide a compound of formula (V): 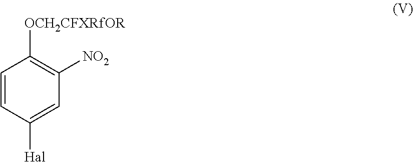
wherein R is a (per)fluoroalkyl group containing from 1 to 3 carbon atoms or a group of formula (VI): 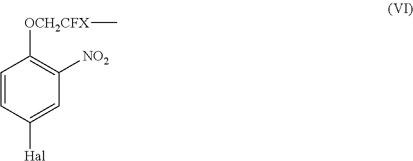
and
condensing, by Suzuki condensation, the compound of formula (VI) with benzene boronic or biphenyl boronic acid to provide a compound of formula (Ia).

11. A process for the manufacture of the (per)fluoropolyether compound of claim 9, the process comprising:
condensing, by Suzuki condensation, a compound of formula (VII): 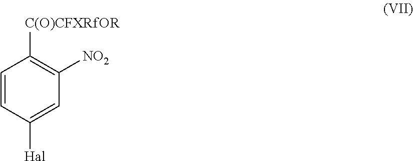
wherein R_{f} is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O— wherein R¹ and R², equal to or different from one another, represent hydrogen, chlorine, a (per)fluoroalkoxy group or a (per)fluoroalkyl group;
X is fluorine or —CF₃;
Hal is a halogen selected from fluorine, chlorine, bromine and iodine;
and R is a (per)fluoroalkyl group containing from 1 to 3 carbon atoms or a group of formula (VIII) 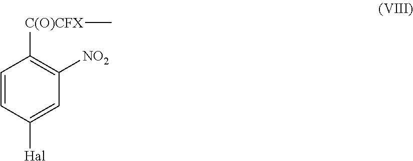
with benzene boronic or biphenyl boronic acid to provide a compound of formula (IX):
R—O—Rᵣ—CFX—C(O)—Ar (IX)
wherein R is a (per)fluoroalkyl group containing from 1 to 3 carbon atoms or a group of formula (X):
Ar—C(O)—CFX— (X)
wherein Ar is selected from:
a biphenyl group of formula (IIa): 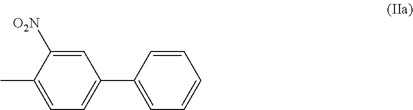 [SMILES: Cc1ccc(-c2ccccc2)cc1[N+](=O)[O-]]
and a terphenyl group of formula (IIb): 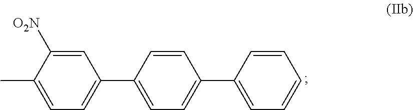 [SMILES: Cc1ccc(-c2ccc(-c3ccccc3)cc2)cc1[N+](=O)[O-]]
and
fluorinating the compound of formula (X) to provide a compound of formula (Ib).

12. A process for lubricating an item, the process comprising contacting the item with a lubricant composition comprising a (per)fluoropolyether compound according to claim 1.

13. A lubricant composition which comprises:
(i) one or more (per)fluoropolyether compounds according to claim 1;
(ii) a (per)fluoropolyether oil;
(iii) optionally, one or more thickening agents and
(iv) optionally, one or more additives.

14. The lubricant composition according to claim 13, wherein the one or more (per)fluoropolyether compound is a compound of formula (Ia) or (Ib):
R—O—R_{f}—CFX—CH₇O—Ar (Ia);
R—O—R_{f}—CFX—CF₂—Ar (Ib);
wherein:
R_{f} is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O— wherein R¹ and R², equal to or different from one another, represent hydrogen, chlorine, a (per)fluoroalkoxy group or a (per)fluoroalkyl group;
X is fluorine or —CF₃; and
R is a (per)fluoroalkyl group containing from 1 to 3 carbon atoms or a group of formula Ar—OCH₂—CFX— wherein Ar is selected from:
a biphenyl group of formula (IIa): 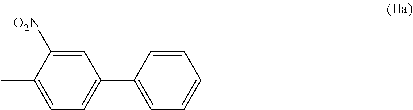 [SMILES: Cc1ccc(-c2ccccc2)cc1[N+](=O)[O-]]
and a terphenyl group of formula (IIb):

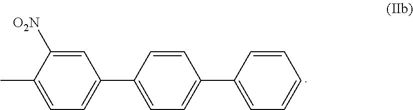 [SMILES: Cc1ccc(-c2ccc(-c3ccccc3)cc2)cc1[N+](=O)[O-]]

15. A lubricant composition which comprises:
(i*) one or more (per)fluoropolyether compounds according to claim 1;
(iii*) one or more thickening agents and;
(iv*) optionally, one or more additives.

16. The lubricant composition according to claim 15, wherein the one or more (per)fluoropolyether compound is a compound of formula (Ia):
R—O—R_{f}—CFX—CH₂O—Ar (Ia)
wherein:
R_{f} is a fully or partially fluorinated polyoxyalkylene chain comprising repeating units R°, statistically distributed along the chain, said repeating units being selected from the group consisting of: —CFXO—, —CF₂CF₂O—, —CF₂CF₂CF₂O—, —CF₂CF(CF₃)O—, —CF(CF₃)CF₂O—, —CF₂CF₂CF₂CF₂O—, and —CR¹R²CF₂CF₂O— wherein R¹ and R², equal to or different from one another, represent hydrogen, chlorine, a (per)fluoroalkoxy group or a (per)fluoroalkyl group;
X is fluorine or —CF₃; and
R is a (per)fluoroalkyl group containing from 1 to 3 carbon atoms or a group of formula Ar—OCH₂—CFX— wherein Ar is selected from:
a biphenyl group of formula (IIa): 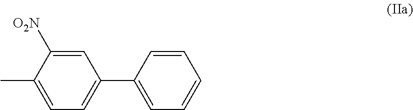 [SMILES: Cc1ccc(-c2ccccc2)cc1[N+](=O)[O-]]
and a terphenyl group of formula (IIb):

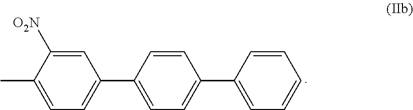 [SMILES: Cc1ccc(-c2ccc(-c3ccccc3)cc2)cc1[N+](=O)[O-]]

17. The lubricant composition according to claim 14, wherein the composition has a kinematic viscosity of between 30 and 3,000 cSt, as determined at 20° C. according to ASTM D445.

18. The lubricant composition according to claim 16, wherein the composition has a kinematic viscosity of between 30 and 3,000 cSt, as determined at 20° C. according to ASTM D445.
